(19) United States
(12) Patent Application Publication
(10) Pub. No.: US 2003/0170223 A1
(43) Pub. Date: Sep. 11, 2003

(54) Pulmonary vasodilator surfactant compositions and method of use

(75) Inventor: Magdy Ahmad, East Lansing, MI (US)

Correspondence Address: MCLEOD & MOYNE, P.C., 2190 COMMONS PARKWAY, OKEMOS, MI, 48864, US

(73) Assignee: Board of Trustees of Michigan State University, East Lansing, MI

(21) Appl. No.: 10/356,062

(22) Filed: Jan. 31, 2003

Related U.S. Application Data
(60) Provisional application No. 60/353,149, filed on Feb. 1, 2002.
(60) Provisional application No. 60/376,293, filed on Apr. 29, 2002.

(51) Int. Cl. A61K 38/43; A61K 33/00; A61K 31/557

(52) U.S. Cl. 424/94.1; 424/718; 514/269; 514/509; 514/573

(57) ABSTRACT

Compositions and methods for use for treating, pulmonary hypertension, in particular neonatal pulmonary hypertension and early lung inflammation, are described. In a first embodiment, the composition comprises pulmonary vasodilators and mitochondrial targeted antioxidants in mixture with a lung surfactant. Preferably, the composition comprises one or more chemicals selected from the group consisting of a long-acting nitric oxide donors, antioxidants, endothelin-1 receptor antagonists, and antithromboxanes in mixture with a lung surfactant. In a second embodiment, the present invention provides a composition for treating neonatal pulmonary hypertension and early lung inflammation in mammals who do not respond to nitric oxide treatment alone. In this embodiment, the composition comprises liposomes comprising a pulmonary or lung surfactant and which contains vasodilators and chemicals which modulate development of acute inflammation. In a preferred embodiment, the liposome contains a long-acting nitric oxide donor, a recombinant human IκB-α, an NF-κB inhibitor, an anti-TNF-α, and an iNOS inhibitor. The composition can further include one or more of the components comprising the first embodiment.

### CROSS-REFERENCE TO RELATED APPLICATION

This application claims benefit of U.S. Provisional Patent Application No. 60/353,149 filed Feb. 1, 2002, and U.S. Provisional Patent Application No. 60/376,293 filed Apr. 29, 2002.

### STATEMENT REGARDING FEDERALLY SPONSORED RESEARCH OR DEVELOPMENT

Not applicable.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to compositions and methods for use for treating-pulmonary hypertension in mammals, in particular, neonatal pulmonary hypertension and early lung inflammation. In a first embodiment, the composition comprises pulmonary vasodilators and mitochondrial targeted antioxidants in mixture with a lung surfactant. Preferably, the composition comprises one or more chemicals selected from the group consisting of a long-acting nitric oxide donors, antioxidants, endothelin-1 receptor antagonists, and antithromboxanes in mixture with a lung surfactant. In a second embodiment, the present invention provides a composition for treating neonatal pulmonary hypertension and early lung inflammation in mammals who do not respond to nitric oxide treatment alone. In this embodiment, the composition comprises liposomes comprising a pulmonary or lung surfactant and which contains vasodilators and chemicals which modulate development of acute inflammation. In a preferred embodiment, the liposome contains a long-acting nitric oxide donor, a recombinant human IκB-α, an NF-κB inhibitor, an anti-TNF-α, and an iNOS inhibitor. The composition can further include one or more of the components comprising the first embodiment.

(2) Description of Related Art

Most of the vasodilators used in treatment of pulmonary hypertension in adults and infants have systemic hypotensive effects in addition to their pulmonary vasodilative effect. These systemic hypotensive effects have restricted the use of vasodilators, particularly in the newborn who in many cases have decreased systemic blood pressure (BP) due to sepsis or severe hypoxia. Because of this, there is a need for a means for selectively targeting pulmonary vasodilators to the lung. This need has been hampered by the lack of a complete understanding of the oxygen sensing process in the neonatal lung.

Regulation of vascular muscle tone at the cellular level appears to occur via nitric oxide (NO), which is most likely identical to the previously-described endothelium-derived relaxing factor (EDRF). Nitric oxide is generated enzymatically by one of several NO synthases from L-arginine. NO activates guanyl cyclase by binding to its heme component, leading to the production of cyclic GMP. The mechanism by which cyclic GMP relaxes vascular smooth muscle is not clear, but most likely involves inhibition of activation-induced elevation in cytosolic calcium concentration.

Abman et al. (______ (1990)) studied the late gestation ovine fetus and showed that inhibition of EDRF production caused fetal pulmonary and systemic hypertension with attenuation of the rise in pulmonary blood flow at delivery. They also demonstrated that inhibition of NO formation attenuated the pulmonary vasodilatation produced by ventilation, increasing oxygen tension and that inhalation of NO by the ovine fetus caused sustained and selective pulmonary vasodilatation of a magnitude equal to that produced by ventilation with 100% oxygen (Kinsella et al., ______ (1992)).

In the newborn lamb, several models of pulmonary hypertension were reversed by 40 to 80 ppm of NO inhaled as a gas (Frostell et al., ______ (1991)). Similar results using pulmonary vasoconstriction produced by hypoxia with or without respiratory acidosis were obtained by Roberts et al. (______ (1993)) who also demonstrated elevated plasma cyclic GMP levels following NO inhalation. In all of these studies, there was no effect on the systemic circulation, specifically on systemic vascular resistance; and in two studies, an increase in left to right shunting across the ductus arteriosus occurred during NO inhalation, demonstrating that inhaled NO (iNO) is a potent selective pulmonary vasodilator.

In adults with Acute Adult Respiratory Distress Syndrome (ARDS), Rossaint et al. (______ (1993)) showed that a reduction in pulmonary arterial pressure and a decrease in intrapulmonary shunting occurred within 40 minutes of inhalation of NO. In these patients, the major benefit was likely due to an improvement in ventilation/perfusion matching, rather than pure pulmonary vasodilatation.

However, notwithstanding the lack of understanding of the oxygen sensing process, it has been found that nitric oxide (NO) is a selective pulmonary vasodilator useful for treating pulmonary hypertension. For example, U.S. Pat. No. 5,570,683 to Zapol, U.S. Pat. No. 5,823,180 to Zapol, and U.S. Pat. No. 5,873,359 to Zapol et al. relate to methods and devices for treating pulmonary vasoconstriction using gaseous NO and a phosphodiesterase inhibitor. NO has recently been approved by the U.S. Food and Drug Administration (FDA) for treating neonatal pulmonary hypertension. However, apart from technical difficulties in administering NO to the patient, as high as 50% of the neonatal cases either do not respond to NO or are poor responders to NO. Thus, there is a need for compositions which are easy to administer and which are more efficacious than NO alone.

U.S. Pat. Nos. 4,826,821 and 4,826,821, both to Clement, relate to lung surfactant compositions which can be administered intratracheally to a patient to treat respiratory distress syndrome. U.S. Pat. Nos. 6,255,354 and 6,228,891, both to Enzmann, describe that for the oral treatment of diseases of the entire digestive tract (mouth, esophagus, stomach and intestine), the effectiveness and the shelf life of pulmonary surfactant can be increased by an addition of at least 0.1% by weight 2,3-dimethoxy-5-methyl-6-decaprenyl-1,4-benzoquinone (coenzyme Q₁₀). Lung surfactant has been used as a vehicle for delivering various chemicals to a patient to treat a variety of diseases as exemplified by the following U.S. patents.

U.S. Pat. No. 6,180,596 to Tsao relates to a method for treating various lung ailments such as cystic fibrosis and other chronic lung diseases by inhibiting the activity of phospholipase A₂ (PLA₂) and suppressing the activity of PLA₂ stimulators in the airway of the patient. The method comprises administering to the patient a composition which in one embodiment comprises annexin I or annexin VIII in a lung surfactant.

U.S. Pat. No. 6,221,856 to Traynor-Kaplan et al. relates to compositions comprising inositol derivatives which in particular embodiments can be mixed with lung surfactant for delivery to the lung. The compositions are useful for preventing or treating in mammals conditions such as inflammation that cause superoxide anion production, oxidative damage of tissues, and pathological conditions caused by superoxide anion production.

U.S. Pat. No. 6,436,970 to Hafner et al. relates to a composition for the treatment of IRDS (Infant Respiratory Distress Syndrome) and ARDS (Adult Respiratory Distress Syndrome) comprising a novel phosphodiesterase IV inhibitor in a lung surfactant.

U.S. Pat. No. 6,459,003 to Dasseux et al. relates to compositions for treating diseases such as hypertension using compounds comprising novel ethers which can be administered to a patient via lung surfactant.

There remains a need for compositions for treating pulmonary hypertension, particularly neonatal hypertension and early inflammation, which is more efficacious than current compositions and which selectively target the lung. There is a particularly acute need for compositions for treating pulmonary hypertension in adults and infants who do not respond to NO treatments which selectively target the lung.

### SUMMARY OF THE INVENTION

The present invention provides compositions and methods for use for treating pulmonary hypertension in mammals, in particular, neonatal pulmonary hypertension and early lung inflammation.

In a first embodiment, the composition comprises pulmonary vasodilators and mitochondrial targeted antioxidants in mixture with a lung surfactant. Preferably, the composition comprises one or more chemicals selected from the group consisting of a long-acting nitric oxide donors, antioxidants, endothelin-1 receptor antagonists, and antithromboxanes in mixture with a lung surfactant.

In a second embodiment, the present invention provides a composition for treating neonatal pulmonary hypertension and early lung inflammation in mammals who do not respond to nitric oxide treatment alone. In this embodiment, the composition comprises liposomes comprising a pulmonary or lung surfactant and which contains vasodilators and chemicals which modulate development of acute inflammation. In a preferred embodiment, the liposome contains a long-acting nitric oxide donor, a recombinant human IκB-α, an NF-κB inhibitor, an anti-TNF-α, and an iNOS inhibitor. The composition can further include one or more of the components comprising the first embodiment.

Therefore, the present invention provides a composition comprising a vasodilator, an antioxidant, and at least one chemical selected from the group consisting of endothelin-1 receptor antagonists and antithromboxanes in a mixture with a pulmonary or lung surfactant. In a further embodiment, the composition comprises a vasodilator, an antioxidant, an endothelin-1 receptor antagonist, and an antithromboxane in a mixture with a pulmonary or lung surfactant.

In further embodiments of the above compositions, the vasodilator is a long acting nitric oxide donor. In particular embodiments, the vasodilator is selected from the group consisting of isosorbide dinitrate, hydralazine, minoxidil, nitric oxide gas, and combinations thereof. In further embodiments, the antioxidant is coenzyme Q₁₀ and in further still embodiments the antithromboxane is selected from the group consisting of prostacyclin.

The present invention further provides a method for treating pulmonary hypertension in a patient, particularly an infant, which comprises (a) providing a composition comprising a vasodilator, an antioxidant, and at least one chemical selected from the group consisting of endothelin-1 receptor antagonists and antithromboxanes in a mixture with a pulmonary or lung surfactant; and (b) administering the composition intratracheally to the patient to treat the pulmonary hypertension. In a further embodiment, the method comprises (a) providing a composition comprising a vasodilator, an antioxidant, an endothelin-1 receptor antagonist, and an antithromboxane in a mixture with a pulmonary or lung surfactant; and (b) administering the composition intratracheally to the patient to treat the pulmonary hypertension.

In further embodiments of the above methods, the vasodilator is a long acting nitric oxide donor. In particular embodiments, the vasodilator is selected from the group consisting of isosorbide dinitrate, hydralazine, minoxidil, nitric oxide gas, and combinations thereof. In further embodiments, the antioxidant is coenzyme Q₁₀ and in further still embodiments the antithromboxane is selected from the group consisting of prostacyclin, and sulotroban.

The present invention further provides a composition which comprises a liposome comprising a pulmonary or lung surfactant and which contains therein a vasodilator and chemicals which modulate development of acute inflammation. The composition is particularly useful for administering to a patient, particularly an infant, who has pulmonary hypertension which does not respond to nitric oxide treatment.

In further embodiments of the composition, the vasodilator is a long-acting nitric oxide donor. In further still embodiments, the chemicals which modulate development of acute inflammation comprise recombinant IκB-α, an inhibitor of activation of NF-κB, anti-TNF-α, and an iNOS inhibitor and peroxynitrite scavenger. In further still embodiments, the anti-TNF-α is a recombinant polypeptide comprising the TNF-α receptor. In further still embodiments, the iNOS inhibitor and peroxynitrite inhibitor is mercaptoethylguanidine. In further still embodiments, the inhibitor of activation of NF-κB is a dithiocarbamate. In further still embodiments, the composition further comprises an antioxidant, an endothelin-1 receptor antagonist, and an antithromboxane in a mixture with a pulmonary or lung surfactant.

The present invention further provides a method for treating pulmonary hypertension in a patient, which comprises (a) providing a liposome comprising a pulmonary or lung surfactant and which contains therein a vasodilator and chemicals which modulate development of acute inflammation; and (b) administering the liposome to the patient intratracheally to treat the pulmonary hypertension. The method is particularly useful for treating a patient, particularly an infant, who has pulmonary hypertension which does not respond to nitric oxide treatment

In further embodiments of the method, the vasodilator is a long-acting nitric oxide donor. In further still embodiments, the chemicals which modulate development of acute inflammation comprise recombinant IκB-α, an inhibitor of activation of NF-κB, anti-TNF-α, and an iNOS inhibitor and peroxynitrite scavenger. In further still embodiments, the anti-TNF-αis a recombinant polypeptide comprising the TNF-α receptor. In further still embodiments, the iNOS inhibitor and peroxynitrite inhibitor is mercaptoethylguanidine. In further still embodiments, the inhibitor of activation of NF-κB is a dithiocarbamate. In further still embodiments, the composition further comprises an antioxidant, an endothelin-1 receptor antagonist, and an antithromboxane in a mixture with a pulmonary or lung surfactant.

### OBJECTS

It is an object of the present invention to provide compositions for treating pulmonary hypertension, particularly neonatal hypertension and early inflammation, which is more efficacious than current compositions.

It is a further object of the present invention to provide compositions for treating pulmonary hypertension in adults and infants who do not respond to NO treatments.

It is a further object of the present invention that the above compositions selectively target the lung.

These and other objects of the present invention will become increasingly apparent with reference to the following drawings and preferred embodiments.

### DESCRIPTION OF THE DRAWINGS

FIG. 1 shows the pathogenesis of pulmonary hypertension in patients who do not respond well or at all to NO therapy.

FIG. 2 shows ammonium sulfate loading procedure for weak bases. Liposomes are first prepared in the presence of 250 mM ammonium sulfate. On removal of the exterior ammonium sulfate on a size-exclusion column, doxorubicin (DOX) is added to the extra-liposomal media. Ammonium sulfate can dissociate to two ammonium cations and one sulfate anion. Ammonia is free to cross the liposomal membrane, giving rise to a pH gradient across the membrane. DOX in its uncharged form can then cross the liposome membrane and form an insoluble gel under acidic conditions with the remaining sulfate anion cooling, effectively trapping it in the liposomal interior. The concentration of DOX in the liposomal lumen can reach concentrations in excess of the aqueous solubility of DOX. This loading procedure can be applied to a variety of weak bases, such as those composing the anthracyclines, Vinca alkaloidam, or camphothercins. However, the stability of the complex formed with sulfate and thus, the gel in the liposomal lumen may help determine the overall stability of the formulation.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

Most of the vasodilators used in treatment of pulmonary hypertension have systemic hypotensive effects together with their pulmonary vasodilative effect. Systemic hypotensive effect of vasodilators usually limit their use, particularly in the newborn who in many cases have decreased systemic blood pressure (BP) due to sepsis or severe hypoxia. Nitric oxide (NO) is a selective pulmonary vasodilator which has been recently approved by the U.S. Food and Drug Administration for treating neonatal pulmonary hypertension. However, apart from its technical difficulties, as high as 50% of the neonatal cases are not responding or are poor responders to NO. Considering that the pathogenesis of neonatal pulmonary hypertension is multifactorial, other drugs such as specific antioxidants beside vasodilators should be added to treat this pathology.

The present invention provides compositions and methods for use for treating pulmonary hypertension and early lung inflammation in patients. The compositions comprise a combination of chemicals comprising at least one pulmonary vasodilator and at least one mitochondrial targeted antioxidant in mixture with a pulmonary or lung surfactant. The composition is administered intratracheally to the patient. By using the pulmonary or lung surfactant as a carrier or vehicle for the chemicals enables the chemicals to be targeted directly to the lung. Targeting the chemicals to the lung avoids many of the adverse effects which might be induced by chemicals used for treating pulmonary hypertension but which are administered systemically because of a perceived lack of the availability of a means for targeting the chemical to the lung. The composition is particularly useful for treating neonatal hypertension in infants, including newborns.

In newborns, successful transition from the intra-uterine low PO₂ environment to the extra-uterine environment is most likely associated with the stimulation of the pulmonary oxygen sensors and the release of pulmonary vasodilators (NO, prostacyclin) after delivery. Failure of this transition may lead to persistent neonatal pulmonary hypertension, a life threatening condition (Kanduri, ______ (2001).

Hypoxia plays an important role as a trigger of pulmonary vasoconstriction. Wayapa et al. demonstrated that mitochondria are the oxygen-sensors in the pulmonary vessels. They showed that hypoxic pulmonary vasoconstriction (HPV) is blocked by agents that inhibit mitochondrial electron transport, absent in mutant arterial myocytes without mitochondria, and inhibited by antioxidants.

Associated etiologies with neonatal pulmonary hypertension include about 49% associated with meconium aspiration syndrome (MAS) (Short B), about 34% due to sepsis and severe respiratory distress syndrome (RDS), and about 17% idiopathic (New England Journal Of Medicine (NEJM)).

Diagnosis of neonatal pulmonary hypertension consists of ECHO's findings of high pulmonary vascular resistance (PVR) and intra- and extra-cardiac right to left shunting together with persistent low PO₂ (less than about 60 Torr in 100% FiO₂) after exclusion of cyanotic heart diseases (NEJM). In most of the clinical identifications of neonatal pulmonary hypertension, the oxygenation index (OI) is less than about 20. Furthermore, in many cases of uncontrolled pulmonary hypertension, the disease might be further complicated with severe hypoxia and multi-organ failure. In many cases of uncontrolled pulmonary hypertension, the case will also include complications from severe hypoxia and multi-organ failure.

The pathogenesis of pulmonary hypertension is as follows. The absence of the pulmonary vasodilators and/or the increased levels of cytokines and endotoxin may be associated with increased reactive oxygen species (ROS) (superoxide anion), reactive nitrogen species (RNS) (nitroxyl anion), or endothelin-1 which will eventually lead to any of the following mechanisms: (1) increasing peroxynitrite production which results in an increase in nitroxyl anion, nitrolysation of the intracellular protein, and increased super oxide anion production (Okayama et al. ______ (1999); (2) decreasing cyclic GMP (cGMP) production which results in an increase Ca⁺⁺ influx into vascular smooth muscles which causes vascular smooth muscle contraction (VSMC); (3) impairment of smooth muscles' mitrochondrial electron transport chain and disruption of coenzyme Q functions which results in depolarization of the vascular smooth muscle membrane and again increased Ca⁺⁺ influx into the cytosol and stimulation of Ryanoidene receptors II leading to VSMC and eventually, pulmonary hypertension; and (4) a similar mechanism may also happen by accumulation of NADH and cADPR (cyclic ADP-ribose) which will stimulate Ryanoidene Receptors by increasing intracellular Ca⁺⁺ leading to VSMC contraction (Dipp et al. ______ (2001)). The composition of the present invention provides an effective treatment for newborns with pulmonary hypertension by abrogating the pathogenesis.

In a preferred embodiment for treating adult and newborn patients, the vasodilator is long-acting nitric oxide (NO) donor such as isosorbide dinitrate, nitric oxide gas, and the like. NO donors can also include gaseous nitric oxide such as described in U.S. Pat. Nos. 5,570,683; 5,823,180; and 5,873,359, all to Zapol et al. In the preferred embodiment, the antithromboxane is a drug such asprostacyclin, sulotroban, and the like; the antioxidant is coenzyme Q₁₀ (also known as ubiquinone, CoQ₁₀, or CoQ); and the composition further includes at least one endothelin-1 receptor antagonist, in mixture with the pulmonary or lung surfactant. In a most preferred embodiment, the composition comprises at least one each of a vasodilator, an antithromboxane, an endothelin-1 receptor antagonist, and an antioxidant in mixture with the pulmonary or lung surfactant. In further embodiments, the above compositions can further include one or more of compounds selected from the group consisting of phosphodiesterase inhibitors, inositol derivatives, ether compounds, and annexins.

Pulmonary or lung surfactant is a surface-active phospholipid-protein material that lines the alveolar epithelium. It reduces the surface tension at the air/water interface, which stabilizes the lung epithelial cells during expiration. Deficiencies of pulmonary surfactant result in respiratory distress syndrome (RDS) in premature infants. The main phospholipids, dipalmitoylphosphatidylcholine (DPPC) and dipalmitoylphosphatidylglycerol (DPPG), are considered to be the principal tensoactive components of pulmonary surfactants. It has also been proposed that surfactant protein-B (SP-B) and surfactant protein-C (SP-C) enhance the surface properties of the surfactant. However, there the role of the pulmonary surfactant constituents at the molecular level are as yet unknown.

Many of the pulmonary or lung surfactants which have been used for the treatment of respiratory distress syndrome in adults and infants are derived from natural sources. For example, U.S. Pat. No. 6,172,203 to Hager et al. discloses methods for extracting and purifying surfactant from lungs of animals. An extract of lung surfactant from pigs is commercially available from Dey, L. P., Napa, Calif., under the trademark CUROSURF and an extract of surfactant from cows is commercially available from Ross Products Division of Abbott Laboratories, Columbus, Ohio, under the trademark SURVANTA. Synthetic pulmonary or lung surfactants are also known in the art. For example, synthetic pulmonary or lung surfactants have been described in U.S. Pat. Nos. 4,826,821 and 5,110,806 to Clements, U.S. Pat. No. 6,315,983 to Eistetter, and U.S. Pat. No. 6,255,354 and 6,228,891 to Enzmann. Both naturally-derived such as CUROSURF or SURVANTA and synthetic pulmonary or lung surfactants such as those disclosed in the above U.S. patents can be used as the vehicle or carrier in the present invention.

When the above compositions of the present invention are administered to the patient intratracheally through an ET tube, the compositions are more efficacious in reducing pulmonary hypertension in an infant or adult than is nitric oxide donor alone. Furthermore, because the compositions of the present invention are targeted to the lung, the compositions can include nitric oxide donors other than nitric oxide gas, such as systemic nitric oxide agents. Nitric oxide donors administered to the patient as a component of the composition of the present invention do not appear to produce the systemic side-effects which might occur when these donors are administered to a patient systemically. Compositions of the present invention which comprise nitric oxide donors other than gaseous nitric oxide are particularly useful for use in medical facilities or clinics which are not equipped to treat pulmonary hypertension using inhaled NO (iNO). Thus, the scope of venues in which pulmonary hypertension, including neonatal pulmonary hypertension and early lung inflammation, can be treated is substantially increased over the venues available when the treatment relies upon iNO.

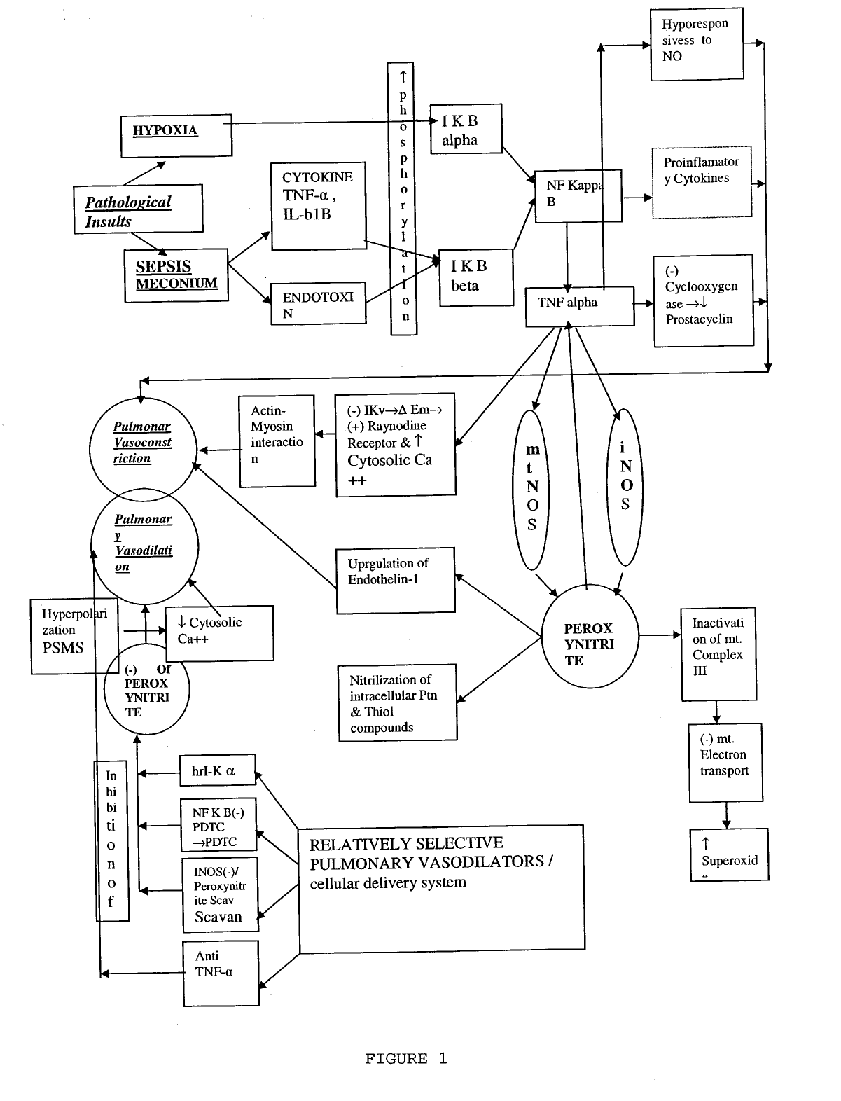

A number of patients with pulmonary hypertension do not respond to inhaled NO (iNO) or systemic nitric oxide agents. A few reports have recently suggested that the inability of iNO to help these non-responders may be due to a defect in the non-responders' NO-cGMP pathway. Wang et al. ______ (2001) reported that administration of TNF-α in vivo depressed endothelium dependent relaxation which suggested a role for NO in this process. The pathogenesis of this form of pulmonary hypertension is illustrated in FIG. 1.

In cases of severe neonatal sepsis or meconium aspiration, the associated endotoxin and proinflammatory cytokines (IL-1β, TNF-α) lead to phosphorylation and degradation of the inhibitory protein kappaB beta (IκB-β), excessive release of nuclear factor kappa (NF-κB), and more increased TNF-α production. On the other hand, in cases of severe neonatal hypoxia, there is excessive degradation of IκB-α and excessive release of NF-κB. A significant elevation in TNF-α level in both situations is associated with up-regulation of the genes controlling IL-6 and inducible nitric oxide synthase (iNOS).

The increase in iNOS production is associated with an increase in cytosolic peroxynitrite in the vascular smooth muscles (VSMC) manifested by increasing super-oxide anion production, nitrolysation of the intracellular proteins and intracellular thiol compounds, and an increase in mitochondrial NOS (mtNOS) with a resultant increase in mitochondrial peroxynitrite which causes impairment of smooth muscles' mitochondrial electron transport chain, depolarization of the vascular smooth muscle membrane, and increased intra-cellular Ca⁺⁺ influx. This may also be associated with stimulation of Ryanoidene receptors II and activation of calmodulin-mediated activation of myosin light chain kinase and actin-myosin interaction and contraction which lead to vascular smooth muscle contraction (VSMC) and eventually pulmonary hypertension.

Increased IL-6 may be associated with decreased prostacycline levels through inhibition of the cyclooxygenase enzyme thereby tipping the balance towards the vasoconstrictor thromboxane A2 and relatively diminishing the pulmonary vasodilator effect. Preliminary data in neonatal sepsis showed increased IL-6 levels with pulmonary hypertension as evidenced by ECHO findings.

NF-κB regulates the expression of many genes in which early response products are critical for the development of acute inflammation. In unstimulated cells, NF-κB is retained in the cytoplasm and bound to inhibitory proteins of the IκB family including IκB-α. In response to inflammatory stimuli, interleukin-1B and reactive oxygen species, IκB-α is phosphorylated by specific kinases and degraded by proteasomes. Degradation of IκB-α releases NF-κB and allows translocation to the nucleus, where NF-κB binds specific promoter elements and induces gene transcription. NF-κB acts on target genes for proinflammatory cytokines, chemokines, immunoreceptors, cell adhesion molecules, and inducible nitric oxide synthase. The activation of NF-κB, therefore, leads to a coordinated increase in the transcription of many genes in which products mediate inflammatory responses. The inhibition of the translocation and transcription requires the presence of cell permeable inhibitory protein (IP) motif for efficient cellular entry of impermeable molecules such as proteins and nucleic acids. In addition, products of the genes that are regulated by NF-κB often cause the activation of NF-κB, which creates a positive feedback loop that may amplify and perpetuate local inflammatory responses.

Hypo-responsiveness of the systemic endothelia to vasodilators such as NO has been proposed to be due to the effect of TNF-α, by several mechanisms such as increased reactive oxygen species and/or inhibition of eNOS. Accordingly, we hypothesize that it has the same effect on pulmonary arteries.

TNF-α is also reported to potentiate the early pathological changes associated with pulmonary interstitial emphysema (PIE) in mechanically ventilated newborn.

We hypothesize that newborns with neonatal pulmonary hypertension that are not responding to iNO probably have significant increase in TNF-α level. This high level will cause up-regulation of specific cytokine genes controlling pulmonary vasomotor tone and at the same time hypo-responsiveness of the pulmonary endothelia to pulmonary vasodilators such as NO. We predict that selective delivery of chemicals which modulate development of acute inflammation (anti-inflammatory drugs) such as pulmonary transcriptional modulators, which affect the transcription of the above genes, and antioxidants can relieve the associated pulmonary hypertension.

Most of the vasodilators used in the treatment of pulmonary hypertension have systemic hypotensive side effects which limits their use, particularly in the newborn who already have in many cases decreased systemic blood pressure (BP) due to sepsis or severe hypoxia. Using selective pulmonary vasodilators such as iNO or other nitric oxide donors to treat pulmonary hypertension has been a compelling idea; however, their use has been hampered by the lack of significant responses in about 50% of the subjects. While iNO has been recently approved by FDA for treating neonatal pulmonary hypertension, there remain many technical difficulties which has limited its use in the medical community. Considering that the pathogenesis of neonatal pulmonary hypertension is multi-factorial, providing a composition comprising specific antioxidants and particular anti-inflammatory drugs and targeting the composition to the lung using pulmonary or lung surfactant as a carrier or vehicle as taught herein provides a successful means for treating these pathologies.

In light of the above, a composition for treating pulmonary hypertension in NO non-responders would include an inhibitor of NF-κB, an inhibitor of degradation of IκB-α, and inactivator of TNF-α, and an iNOS and peroxynitrite scavenger.

Thus, one of the chemicals for including in the composition is recombinant human IκB-α which can maintain the inhibitory effect on NF-κB and hence control the release of TNF-α. It is more selective than the newly described proteasomes inhibitor. It is a lipophilic compound which has been tested by delivery through a liposomal system. It is poorly soluble in aqueous solutions but can be dissolved in pulmonary or lung surfactant such as the lung surfactant we are currently using to treat surfactant consuming/deficiency which is associated in many cases with pulmonary hypertension. Using pulmonary or lung surfactant as a solubilizing agent/vehicle to deliver other lipophilic drugs to the lung is a novel idea which enables the selective delivery of many different pulmonary chemicals or medicines to the lung and which has not been thoroughly investigated.

A second chemical to include is a dithiocarbamate, such as pyrrolidine dithiocarbamate (PDTC). PDTC is a potent inhibitor of degradation of IκB-β and which has been tested in vivo and in experimental lung transplant preservation media. It improved significantly pulmonary oxygenation, and reduced pulmonary mean arterial pressure (MAP), edema, and cellular infiltration. However, PDTC, like IκB-α, is poorly soluble in aqueous solutions. Thus, PDTC represent a class of antioxidants reported to be potent inhibitors of NF-κB which are capable of inhibiting the inflammatory process associated with the activation of NF-κB. It is considered one of the most effective inhibitor of NF-κB as a result of both ability to traverse the cell membrane and its prolonged stability in solution at physiological pH. PDTC, may also inhibit NF-κB production by intracellular reactive oxygen species.

A third chemical is an etancept (anti-TNF-α). ETANERCEPT is a recombinant TNF receptor that binds to and functionally inactivates TNF-α. Administration of TNF-α has been shown experimentally to depress endothelium-dependent relaxation, and ETANERCEPT was reported to improve the depressed systemic endothelial vasodilator function. Fichtlscherer (______ (2001)) reported that the administration of ETANERCEPT profoundly improves systemic endothelial vasodilator capacity, suggesting an important role of inflammatory mediators for impaired endothelial vasoreactivity. The deficit in peripheral vasodilator capacity at least in part results from attenuated vascular endothelial function and has been attributed to a loss of the ability of the endothelium to release nitric oxide.

TNF-α is a well-established mediator of activation of endothelial cells, which results in impaired vasoreactivity. The mechanism by which TNF-α can acutely impair endothelial function has not been clearly prescribed. Cell culture studies have shown that TNF-α rapidly increases the production of reactive oxygen species, which might inactivate nitric oxide and thereby inhibit endothelium-dependent relaxation. In addition, TNF-α can block activation of endothelial nitric oxide synthase (eNOS) by interfering with Akt phosphorylation, which is essential for ACH-dependent relaxation of blood vessels. Besides the post-transcriptional inactivation of eNOS, TNF-α is further capable of directly degrading eNOS mRNA.

See also the following references: Levine et al., New Engl. J. Med. 323: 236-241 (1990); Kubota et al., Circ. Res. 81: 627-635 (1997); Bozkurt et al., Circulation 103: 1044-1047 (2001); Deswal et al., Circulation 99: 3224-3226 (1999); Harrington and Coats, Curr. Opin. Cardiol. 12: 224-23 (1997); Hirooka et al., Circulation 90: 658-668 (1994); Wang et al., Am. J. Physiol. 266: H2535-H2541 (1994); Fichtlscherer et al., Circulation 102: 1000-1006 (2000); Hornig et al., 97: 363-368 (1998); Chavakis et al., Circulation 103: 2102-2107 (2001); Hermann et al., Arterioscler. Thromb. Vasc. Biol. 20: 402-409 (2000); Dimmeler et al., Nature 399: 601-605 (1999); Luo et al., J. Clin. Invest. 106: 493-499 (2000); Yoshizumi et al., Circ. Res. 73: 205-209 (1993); Rossig et al., J. Am. Coll. Cardiol. 36: 2081-2089 (2000); and, Dimmeler et al., J. Exp. Med. 185: 601-607 (1997).

Since peroxynitrite is a major contributor to the pathology of pulmonary hypertension, inclusion of an iNOS and peroxynitrite scavenger is of great benefit. iNOS inhibitors and peroxynitrite scavengers include mercaptoethylguanidine.

The solubility problem of PDTC and IκB-α is overcome by mixing the PDTC and IκB-α with a pulmonary or lung surfactant as a solubilizing vehicle to produce surfactant liposomes. By further including a vasodilator, an iNOS inhibitor and peroxynitrite scavenger, and an anti-TNF-α chemical in the liposomes, a composition is provided which can then be delivered directly to the lung through an ET tube to relieve pulmonary hypertension in adult and neonatal infants, particularly in adults and neonatal infants who do not respond to NO alone.

Therefore, in a further embodiment of the present invention, the present invention provides a composition which is particularly efficacious for treating patients with pulmonary hypertension but who do not respond to nitric oxide treatment because of a significant increase in TNF-α levels in the patient. The composition is particularly useful for treating infants with neonatal pulmonary hypertension and early inflammation but who do not respond to nitric oxide treatment. The composition comprises a liposome comprising a pulmonary or lung surfactant and which contains therein a vasodilator and chemicals which modulate development of acute inflammation.

Preferably, the vasodilator is a long-acting nitric oxide donor and the chemicals are natural or recombinant human I-kappaB-alpha (IκB-α), an inhibitor of activation of NF-κB; an inhibitor of degradation of IκB-αsuch as a dithiocarbamate, preferably a dithiocarbamate such as pyrrolidine dithiocarbonate (PDTC); an inducible nitric oxide synthase (iNOS) inhibitor or peroxynitrite scavenger such as mercaptoethylquanidine (MEG); and, an anti-TNF-α chemical such as Etanceptor which is a recombinant polypeptide comprising the TNF-α receptor and which binds to TNF-α and inactivates it. The composition is administered to the patient intratracheally.

In further embodiments, the liposome composition can further include an antithromboxane, an endothelin-1 receptor antagonist, and an antioxidant in mixture as described above or the liposome composition can be mixed with a composition comprising an antithromboxane, an endothelin-1 receptor antagonist, and an antioxidant mixed with a pulmonary or lung surfactant. The latter can optionally contain a vasodilator. In further still embodiments, any one of the previously described compositions or liposome compositions can further include one or more chemicals such as diphenyleneiodium (DPI), rotenone, myxothiazol, antimycin A, cyanide, ebselen, diethyldithiocarbamate (DDC), apocynin, and 4,4′-diisothiocyanatostilbene-2,2′-disulphonic acid (DIDS) or one or more of the above chemicals in lieu of any one or more of the chemicals described previously.

The general background about developing a pulmonary or lung surfactant liposomal system for the present invention is as follows. Physical stability of liposomal drug formulations is a consideration. For amphipathic drugs that can readily cross membranes, there are a variety of factors that can influence the stability of a liposomal formulation. The presence of cholesterol and saturated phospholipids appear to be the most important factors for reducing membrane permeability of these drugs (Bally et al., ______ (1990); Gabizon et al., ______ (1993)). Other factors, such as the drug-loading method, which can result in internal concentrations of the drug exceeding the aqueous solubility of the free drug, also act to stabilize the formulation. High density liposomes (HDLs) have been shown to destabilize pure PC (phosphatidylcholine) surfactant liposomes by catalyzing the net exchange of PC from surfactant liposomes to the HDL particles (Scherphof et al., ______ (1978); Chobanian et al., ______ (1979); Damen et al., ______ (1980)).

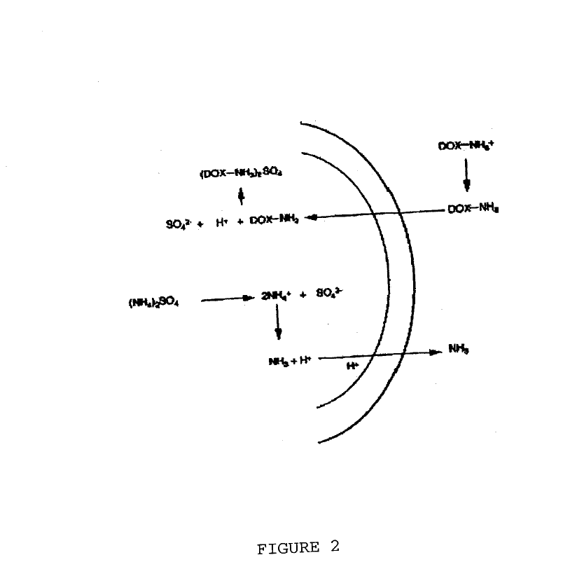

Chemical-loading methods in non-natural surfactant liposomal systems is a consideration. A diagram depicting the ammonium sulfate remote-loading procedure for loading a chemical in liposomes is shown in FIG. 2 (Haran et al., ______ (1993); Lasic et al., ______ (1995)).

Lipids are typically hydrated to form suspensions in high concentrations of ammonium sulfate (250 mM) which are subsequently extruded to produce liposomes of the desired size. Unencapsulated ammonium sulfate is removed (for example using a size-exclusion column), and the drug is added to the surfactant liposomes. Although ammonia can freely pass through membranes in its neutral form, sulfate is trapped in the liposomal lumen. When ammonia moves out of the liposome going with the concentration gradient, a hydrogen ion is left behind and a self-sustaining pH gradient is formed. Chemicals move in their neutral form in the opposite direction and becomes protonated, eventually forming an insoluble salt with the entrapped sulfate anions. The resulting gel helps stabilize the chemical in the interior. The cooling that occurs after the loading step, which is performed at 55-60° C., also plays a role in solidifying the chemical precipitate and thus, increasing the stability of the formulation. A pH gradient strategy for loading weak bases was reported initially by Nichols and Deamer (Biochim. Biophys. Acta 11: 269-217 (1976)) and later used extensively by Cullis (______), with a pH gradient to drive the accumulation of chemicals into surfactant liposomes (Mayer et al., ______ (1985); Madden et al., ______ (1990); Harrigan et al., ______ (1993); Cullis et al., ______ (1997)). Weak acids can be loaded in an analogous manner using calcium acetate or reverse pH gradients (Clerc and Barenholz, ______ (1995) Cullis et al., Biochim. Biophys. Acta. 1331: 187-211 (1997)). These gradients also help stabilize formulations and reduce leakage during storage and while in the circulation.

Chemical to lipid ratio is important. An optimal chemical/lipid ratio is known to be important in the development of a stable formulation. The chemical/lipid ratio should be as high as possible to maximize the payload of chemical reaching the lung without compromising stability. The maximum amount of chemical loaded per surfactant liposome is dependent on the method used for chemical loading, the size of the surfactant liposome, and the presence of trapping components such as acidic lipids to which the chemical can bind. Because the latter two factors are traditionally associated with negative effects on pharmacokinetic parameters, the chemical-loading method is the most readily adjustable method and is preferred.

For passive encapsulation of chemicals in cationic lipids (CLs), the concentration achieved was 0.079 μg chemical/μg lipid. For remote loading via a simple pH gradient, the most effective concentration reached was 0.250 μg chemical/μg lipid, and for remote-loading using an ammonium sulfate gradient, it was 0.125 μg chemical/μg lipid. Chemical/lipid ratios that are too high can form less stable formulations, presumably due to the dissipation of the pH gradient during chemical loading (Mayer et al., ______ (1990); (1993). These results emphasize the care needed in optimizing chemical-loading methods to prepare stable surfactant liposomes and at the same time maximize encapsulation efficiencies.

Stabilizing against aggregation is important. The presence of PEG on the surface of the liposome provides a steric barrier that prevents surfactant liposome aggregation. PEG-coated surfactant liposomes are stable with respect to both size and chemical-encapsulation over the period of many months to years when stored below the phase transition of the PC component (Haran et al., ______ (1993); Lasic and Needham, ______ (1995)).

Stability of chemical and lipid components is important. Questions to be explored include the following. Are the chemicals and lipid components compatible with the remote-loading technique used? If ligand-mediated targeting results in endocytosis of the surfactant liposome, is the chemical stable in the low pH environment of late endosomes and lysosomes or in the presence of degradative enzymes present in these structures?

Lipid peroxidation is another important concern for unsaturated lipid components. Lipid peroxidation can be initiated by a variety of different factors and can lead to the formation of membrane-destabilizing secondary oxidation products such as 4-hydroxynonenal and malondialdehyde (Frankel, ______ (1987); Barenholz et al., ______ (1993)). Phospholipids containing diunsaturated fatty acyl chains such as linoleic, linolenic, or arachidonic acid are particularly susceptible to lipid peroxidation due to the ready abstraction of hydrogen radicals from doubly allylic carbons (Frankel, ______ (1980); ______ (1985)). Linolenate- and arachidonate-containing phospholipids are the most likely to form complex secondary oxidation products which are particularly damaging to membranes (Frankel, ______ (1987)). This brings up an important point concerning the presence of unsaturated lipids. However, in any event, a lipid component with the desired Tₘ can be obtained by balancing the acyl chain length and the number of unsaturations found in a particular phospholipid component.

The stability of a liposomal formulation is dependent on many physical and chemical factors, ranging from the individual chemical and lipid components to the stable encapsulation of the drug within the carrier. A rigorous undertaking is necessary in developing any new liposomal drug formulation to ensure these stability considerations are addressed.

Bioavailability of liposome encapsulated chemicals is important. The bioavailability of a chemical in the liposome is dependent on how readily it is able to escape the liposomal carrier. In the case of liposomal carriers, bioavailability is defined as the amount of free chemical that is able to escape the confines of the carrier and thus, is available for redistribution to neighboring tissues of the lung. For chemical-loaded slow-release surfactant liposomes, the chemical is thought to leak very slowly.

The following examples are intended to promote a further understanding of the present invention.

### EXAMPLE 1

This example illustrates the embodiment of the present invention for treating neonatal pulmonary hypertension and early lung inflammation in infants using a composition comprising a long-acting NO donor vasodilator, an antioxidant, an endothelium-1 receptor antagonist, and an antithromboxane in mixture with a pulmonary or lung surfactant. The present invention is efficacious in neonatal patients.

The main objectives of this example are as follows. First is initiating a base-line for the similarities between animal models of pulmonary hypertension, of those not-responding to NO, and human newborns. Second is investigating the reasons for failure of about 50% of neonates with pulmonary hypertension to respond to the vasodilatation effect of (NO) through evaluating the balances between the different mediators by two approaches: one being molecular biology for assessing the levels and the balances between the different vascular tone mediators (NO, cGMP, peroxynitrite, nitroxyl radicals, endothelin-1 mRNA, and the mRNA of the voltage-gated potassium channels (Kv), voltage-dependent calcium channels (VDCC) (Zapol, Salzman, and Archer, ______ (1998); ______ (1997); ______ (2002) and the other being gene regulation for determining the effect of different inducers or promoters affecting the genes controlling the vascular tone and also exploring the effect of close location proximity of the carbamoyl phosphate enzyme locus and endothelin-1 converting enzyme on chromosome 2q35-36 on the whole picture of pulmonary vascular tone (NEJM). Third, is treating pulmonary hypertension in non-nitric oxide medical facilities with an affordable intervention that covers both nitric oxide responders and non-responders to nitric oxide, without significant systemic side effects, with the following chemicals or medicines delivered to the lung in pulmonary or lung surfactant as a solubilizing material and vehicle for carrying the following chemicals: a long acting nitric oxide donors (such as those described in the Zapol Abstract ______ (1999)), an endothelin-1 antagonist (such as 123 BQ which is available from A.G. Scientific, Inc., San Diego, Calif.), prostacyclin and analogs (an anti-thromboxane) (Olschewski, N. Engl. J. Med. (2002), and an antioxidant, preferably mitochondrial targeted such as CoQ₁₀ (Langsjoen and Langsjoen, BioFactors 9: 273-284 (1999); Pobezhimova and Voinikov, Membr. Cell Biol. 13: 595-602 (2000); Yokoyama et al., Surgery 120: 189-196 (1996); Clarke, ______ (1993)). Fourth, is developing long acting pulmonary tissue specific NO donors similar to the already developed hepatic tissue specific NO donors.

The intervention Strategy for the treatment in this example takes into consideration that because the pathogenesis of neonatal pulmonary hypertension is multifactorial, other drugs such as specific antioxidants beside vasodilators should be added to treat this pathology. One of these drugs is CoQ₁₀ which is an antioxidant that acts at the mitochondria level, and is currently being evaluated at the national Institutes of Health for treatment of adult neurological disorders. However, it has not been evaluated for treating hypertension because it is only lipid soluble which means it cannot be provided to the lungs as a component of an aqueous solution. Previous attempts to produce a soluble chemical with an antioxidant activity similar to CoQ₁₀ have failed (Clarke, In Critical reveiew of Stress, by Gutler 2002). However, CoQ₁₀ has been made soluble in some special types of media (Monroe, ______). We observed that the media that was used contained components similar to those which comprise the pulmonary or lung surfactant we are currently using to treat surfactant consuming/deficiency disease which in many cases is associated with pulmonary hypertension. Recently, it was reported that CoQ₁₀ can also diminish the production of pro-inflammatory mediators (Clarke, ______). Thus, its use as a component of the composition of the present invention can decrease the incidence and severity of neonatal chronic lung disease. In light of the above, we discovered that the solubility problem of CoQ₁₀ can be resolved by solubilizing CoQ₁₀ in pulmonary or lung surfactant. Thus, CoQ₁₀ solubilized in pulmonary or lung surfactant is put in a form which is available for treating pulmonary hypertension.

By mixing the pulmonary or lung surfactant containing CoQ₁₀ with other chemicals or medicines such as long acting nitric oxide donors, selective endothelin-1 receptor antagonists, and antithromboxanes, and delivering the mixture directly to the lung through an endotracheal (ET) tube, management of the multifactorial pulmonary hypertension of neonatal infants can be achieved without significant systemic side effects. Therefore, by mixing CoQ₁₀ with at least one long acting nitric oxide donor, at least one selective endothelin-1 receptor antagonist, and at least one antithromboxane, and then mixing with a pulmonary or lung surfactant, we can not only deliver the mixture locally to the lung through ET tube to fully and effectively treat or manage this multifactorial disease without significant systemic side effects but at the same time resolve the solubility problem of various chemicals such as CoQ₁₀.

Using a rabbit animal models and using isolated perfused lungs, the following steps are performed for evaluating the efficacy of the various components comprising the composition of the present invention. Induction of pulmonary hypertension is by Hypoxia 10% O₂, endotoxin (LPS) injection, or meconium aspiration into ET tube using fine granulated meconium installed into the ET tube. Medical intervention is using one or more, preferably all, of a long-acting NO donor, an anti-endothelin-1 antagonist, CoQ₁₀ (antioxidant), and prostacycline (antithromboxane) mixed with a pulmonary or lung surfactant as the vehicle and delivering the mixture locally to the lung.

Monitoring the response parameters to the interventions with the mixtures and comparing them to parameters obtained before intervention. The following parameters are monitored: (1) local tracheal aspirate for neutrophil counts, IL-6 and IL-8 cytokines (as markers for inflammation), Ahmad, ______ (1997)); (2) cellular/vascular smooth muscles: (a) reactive oxygen species ROS (Superoxide anion), (b) reactive nitrogen species NRS (nitroxyl anion) together with tyrosine nitration of the intra-cellular protein, and (c) cellular nitrolysation by DFC; and (3) mRNA for Kv, endothelin-1 receptors antagonist, and mRNA for NOS 1 and 2.

### EXAMPLE 2

This example illustrates the embodiment of the present invention for the targeted delivery of transcriptional modulators and antioxidants in mixture with a pulmonary or lung surfactant and nitric oxide for treating infants with neonatal pulmonary hypertension and early inflammation who do not respond to nitric oxide treatment.

The main objectives of this example are as follows. First, to investigate the pathological changes (in sequential steps, with single and combined pathological insults) in pulmonary artery endothelia and myocytes, as which happens with pulmonary hypertension. This may elucidate the reasons for failure of about 50% of neonates with pulmonary hypertension to respond to iNO. The levels and the balances between different pulmonary vascular tone modulators: NO, peroxynitrite, TNF-α, NF-κB, and nitroxyl radicals are assessed. Second, to identify the origins of different cytokines controlling the pulmonary vascular tone: infiltrating inflammatory cells, endothelia, or pulmonary myocytes. Third, to provide treatments for neonatal pulmonary hypertension in medical facilities that do not have iNO delivery systems which are affordable and which cover both potential iNO responder and non-responder newborns and which are delivered to the lung with a pulmonary or lung surfactant as a solubilizing media/vehicle, without significant systemic side effect. The primary end point is to increase the OI to greater than 40.

Materials and Methods:

Therapeutic Agents: the composition of the present invention comprising (a) human recombinant IκB-α, (b) transcriptional Modulator: NF-κB inhibitor (pyrrolidine dithiocarbamate, (PDTC)) (c) iNOS inhibitor/Peroxynitrite Scavenger, and (d) Anti TNF-α (ETANERCEPT) in mixture with a pulmonary or lung surfactant vehicle to deliver them locally to the lung.

IκB-α/surfactant preparation: 0.2 mL of SURVANTA (has 2.0 mg of phospatidylcholine) is dissolved in chloroform, and dried in chloroform-pretreated 12×75 mm glass tubes by rotation in a vacuum. Human IκB-α-GST fusion protein (50 μg) is dissolved in 100 μL of 50 mmol Tris-HCl (pH 7.5) and added to the dried lipid. The mixture is agitated by alternate cycles of sanitation (10 seconds) and vortex (20 seconds). Liposomes with GST moiety alone are prepared in a similar way, but IκB-α-GST fusion protein is substituted with an equimolar concentration of recombinant GST. The liposome mixtures are extruded for 20 passes through a 0.1 mm membrane with the aid of an ethanol-pretreated extrusion device and mixed with DMEM/5% FBS media.

The liposomes are also fabricated to contain PDTC, iNOS inhibitors and peroxynitrite scavengers, ETANERCEPT (anti TNF-α), and vasodilators. Other pharmacological and chemical agents such as diphenyleneiodonium (DPI) obtained from CalBiochem and rotenone, myxothiazol, antimycin A, cyanide, ebselen, diethyldithiocarbmate (DDC), apocynin, and 4,4′-diisothiocyanatostilbene-2,2′-disulphonic acid (DIDS) from Sigma can be included in various combinations. In general, the pharmacological agents are dissolved in DMSO (100%) as a 1000×stock solution so that when an aliquot of the stock solution is added to the media, the DMSO concentration is less than about 0.1%. The stock solutions are stored at −20° C. and thawed on the day of the experiment.

Cellular uptake studies are preformed to assess the bioavailablity of drugs delivered to pulmonary vascular smooth muscles.

Alternatives to Cellular Delivery System include using a central venous line to deliver the combinations of drugs to the right atrium of the heart and then to the pulmonary arteries.

Combination Strategies include the following depending on the presumed etiologies for pulmonary hypertension.

(I) Methods in Hypoxic Pulmonary Vasoconstriction

Estimation of wedge pressure in intact animals is performed to determine any possible effect of significant right to left shunting on the pharmacokinetic of the intervention drugs (before and after exposure to pathological insults).

Isolated perfused lung preparation prepared from newborn rats. Male or female newborn (less than 48 hours old) Sprague-Dawley rats are anesthetized with pentobarbital sodium (0.65 mg/kg; ip), and heparin (1000 U/kg) is administered intravenously. Lungs are isolated as follows. Briefly, the lungs and the heart are remove en block and the pulmonary artery (PA) and left atrium are cannulated and perfused with a buffered salt solution (BSS) (NaCl (117 mM), KCl (4 mM), NaHCO₃ (18 mM), MgSO₄ (0.76 mM), NaHPO₄′H₂O (1 mM), CaCl₂ (1.21 mM), and glucose (1 g/L)) containing bovine serum albumin (0.5% w/v) and indomethacin (10 mg/L). After flushing the pulmonary circulation with perfusate (100-200 mL), the lungs are perfused from a recirculating system (35 mL) at 8 mL/min. Perfusate is maintained at 380 C, pH 7.4, and bubbled with 5% O₂, 5% CO₂, and 90% N₂. Left atrial pressure is maintained at 1 cm H₂O₂ by elevating the venous outflow cannula. The lungs are ventilated with a small animal respirator using a humidified gas mixture of 21% O₂, 5% CO₂, and 74% N₂ (normoxia) at a rate of 54 breaths/in, a tidal volume of 2-3 mL and an end-expiratory pressure of 3 cm H₂O₂. Left atrial and PA pressures are continuously recorded on a multichannel strip chart recorder (Grass Instruments, Quincy, Mass.).

HPV responses in isolated lungs from the newborn rats. Angiotensin II (10 nM) is added to the recirculating perfusate because isolated lungs perfused with BSS does not respond to hypoxia without such an agonist. This typically increased PA pressure by 2 cm H₂O. HPV is induced by switching ventilation from the normoxic gas to a hypoxia mixture containing 2% O₂, 5% CO₂, and 93% N₂ for 10 to 15 minutes during which time PA pressure increased. When the PA pressure had stabilized at the higher value, the lungs are returned to normoxia and the pressure in the PA returned to baseline.

Hypoxia-induced changes in pulmonary vascular impedance are represented as the change in PA pressure observed during hypoxic ventilation under constant flow conditions, compared with normoxia, in cm H₂O. Two hypoxic challenges are performed and averaged to define the baseline response prior to the experimental intervention, thus allowing each lung to act as its own control. The experimental agents then are added to the reservoir and allowed to recirculate for 5-10 minutes, after which two more hypoxic challenges are administered and averaged to evaluate the effects of the intervention. In the continued presence of the experimental agents, the stable thromboxane A₂ analog, U46619 (5 ng/mL) is then added to the reservoir to determine the effect on the ability to respond to a receptor-mediated vasoconstricting agonist. This provided a measure of the specificity of the intervention for the response to hypoxia.

Non-specific inhibition of PA smooth muscle cell contraction in the lungs from new born rats. To determine whether the pharmacological agents that inhibited the HPV response in our study might also affect the response a receptor-mediated vasoconstrictor, studies are carried out using the stable Thromboxane A2 analog, U46619 (5 ng/mL). Experiments are performed to assess the U46619 response before and after the addition of the experimental agents. Briefly, lungs are subjected to a challenge of U46619 and the change in PA pressure is measured. The vasculature is then washed out with fresh perfusate until the PA pressure returned to baseline (approximately 10 minutes elapsed time), after which the experimental agents are administered to the reservoir. After 10 minutes, a second dose of U46619 is added to the reservoir and the change in PA pressure is measured. Only Rotenone blunted the U46619 vasoconstriction at 5 μg/mL. Myxothiazol (50 μg/mL), cyanide (10 μM), ebselen (50 μM), and DDC (1 μM) had no effect on the U46619 vasoconstriction response. DPI (10 μM), rotenone (50 ng/mL), antimycin A (1 ng/mL), PDTC (10 μM), and DIDS (200 μM) augmented the U46619 vasoconstriction response. These results indicate that the inhibition of HPV, observed with some of the compounds, is not a reflection of nonspecific inhibition.

Pulmonary microvessel myocyte isolation from the isolated lungs from the newborn rats. Myocytes are isolated using a modification of the method of Marshall et al. (______ (1999). Freshly excised rat heart and lungs are rinsed with phosphate-buffered saline (PBS) containing penicillin and streptomycin (1%). The right ventricle is cannulated and the pulmonary vasculature is flushed with the PBS (30 mL). Using the PA cannula, M199 growth media (30 mL) containing HEPES (25 mM), and penicillin and streptomycin (1%) plus low melting point agarose (0.5%) and iron particles (0.5%), is flushed through the pulmonary vasculature. Because the iron particles are too large to pass through the capillaries, only the arteries contained iron particles. The airways are filled via the trachea with M199 (15 mL) containing low melting point agarose (1%) without iron. The lungs are plunged into cold PBS to cause the agarose to gel. After 10 minutes, the lobes are dissected free and finely minced in a petri dish. The lung fragments are washed (3×) with PBS, using a magnet to retain the iron-containing fragments. The iron-containing pieces are resuspended in M199 media (25 mL) containing collagenase (80 U/mL) and incubated at 37° C. for 30-60 minutes. To remove extra-vascular tissue, fragments are first drawn through a 15-gauge needle, and subsequently through an 18-gauge needle. The iron-containing fragments are then washed (3×) with M199 containing fetal bovine serum (FBS) (20%) and drained. The resulting fragments are placed in a petri dish and resuspended in M199 containing FBS (10%) (4 mL). The petri dishes are incubated at 37° C. with CO₂ (5%) in air for 4-5 days, during which time the pulmonary myocytes are observed to migrate and adhere to the dish. After 4-5 days, the media and iron containing particles are transferred to a new dish containing fresh media. The adherent myocytes continued to propagate until the cells are 70% confluent. The myocytes are then replated on collagen-coated (0.01%) glass cover slips and grown in M199 with FBS (10%) until 70% confluent for measuring ROS generation or 15% confluent for measuring cell shortening.

Immunocytochemistry of the monocyte cultures prepared from the lungs of the newborn rats. To confirm that the cultured cells are indeed pulmonary arterial myocytes, isolated pulmonary smooth muscle cells and A549 cells (for comparison) are grown on coverslips in 6-well plates containing M199 with 10% FBS, penicillin and streptomycin. Before staining the cells, the medium is removed, the cells washed with PBS twice, fixed with 100% methanol at −20° C. for 10 mm, and washed twice with PBS. The cells are then incubated with blocking buffer (5% FBS in PBS) for 1 hour, and incubated with 1:100 dilution of monoclonal anti-smooth muscle cell α-actin antibody (Sigma) in blocking buffer for 1 hour. Subsequently the cells are washed with PBS four times, and incubated with 1:100 dilution of FITC-labeled anti-mouse antibody (Amersham) in blocking buffer for 45 minutes. Finally, cells are washed with PBS four times and mounted with PERMOUNT medium (Fisher). The slides are air dried and examined with a fluorescence microscope. Pulmonary smooth muscle cells stain for a smooth muscle actin, whereas A549 do not stain for a smooth muscle actin. This shows that the cultured cells are indeed pulmonary arterial myocytes.

HPV responses in pulmonary myocytes isolated from the lungs of newborn rats. Glass cover slips containing isolated pulmonary myocytes are placed in a stainless steel flow-through chamber on an inverted microscope and studied under controlled O₂ and temperature conditions. The chamber is sealed using thin-wafer gaskets to minimize any O₂ exchanges between the chamber wall and perfusate. A water-jacketed glass equilibration column (37° C.) mounted above the microscope stage is used to equilibrate the perfusate to known O₂ tensions (PO₂). The perfusate consisted of M199 media without serum. A precision mass flow controller supplies the gas used to control the PO₂ and PCO₂ of the perfusate. In previous studies, the PO₂ in the chamber is confirmed under conditions identical to those of the experiments using an optical phosphorescence quenching method (OXYSPOT, Medical Systems, Inc.). Contraction of pulmonary myocytes is assessed by measuring changes in cell length as described previously. Myocytes on cover slips in the flow-through chamber are visualized using Hoffman Modulation phase contrast optics. Lengths of individual myocytes are measured from computer images. Myocyte contraction at 30 min is expressed as percent decrease from the original length at t=0, ([(original length)−(length at 30 mm)/(original length)]×100).

Pulmonary microvessel endothelia isolation and measurement of reactive oxygen species (ROS). ROS generation in pulmonary endothelia and myocytes is assessed using the probe 2′,7′-dichlorofluorescin diacetate (DCFH-DA, 5 μM Molecular Probes), which is continuously present in the perfusate. The diacetate form enters the cells where esterases cleave the acetate group, tending to trap the nonfluorescent 2′,7′-dichlorofluorescin (DCFH) intracellularly. In the presence of H₂O₂, this probe is oxidized to 2′,7′-dichlorofluorescein (DCF), which is quantified using fluorescence imaging (excitation: 488 nm, emission: 535 nm) in a system equipped with a 12 bit digital camera. Intensity values are reported as percent of initial values, after subtracting background (METAMORPHIC, Universal Imaging, Inc.).

In steady state, increases in fluorescence occur when the rate of intracellular oxidation of the dye is accelerated. Chemical reduction of DCF to DCFH does not occur in cells, so decreases in fluorescence reflect a lowered rate of oxidation of the dye, combined with a leakage of the oxidized dye from the cell. To confirm that the decrease in DCF fluorescence seen upon reoxygenation is not due to a loss of plasma membrane integrity, additional studies are carried out using SYTOX Green dye (100 nM) to detect changes in membrane permeability. PA myocytes are studied during normoxia (15% O₂), during 30 min hypoxia (2% O₂), after return to normoxia, and after the plasma membrane is disrupted using digitonin (300 μM). No increase in nuclear staining is observed during hypoxia or after reoxygenation, but all cells demonstrated nuclear staining with SYTOX Green after digitonin. These findings suggest that loss of membrane integrity cannot explain the fall in intracellular DCF fluorescence observed at deoxygenating.

To confirm that the DCFH dye is sensitive to oxidation by H₂O₂, additional studies are carried out in PA endothelia myocytes superfused with normoxic (16% O₂) media in the presence of DCFH-DA (5 μM). After a steady level of intracellular fluorescence is reached, H₂O₂ is added to the media and the intracellular fluorescence increased significantly and progressively. When the H₂O₂-containing media is replaced with fresh media containing DCFH without H₂O₂, intracellular fluorescence decreased toward baseline values. These findings confirm that DCFH oxidation is sensitive to H₂O₂, and that oxidized DCF can leak out of viable cells and explain the decrease in fluorescence seen at reoxygenation.

To confirm that the loss of DCF fluorescence upon reoxygenation is not due to cell death subsequent to the opening of the mitochondrial permeability transition pore, additional studies are carried out using tetraethyrhodamine methyl ester (TMRE) to assess mitochondrial membrane potential (ΔΨm). This cationic dye equilibrates across the mitochondrial inner membrane in accordance with the AΔΨm, and changes in endothelia fluorescence at these non-quenching concentrations can be used to detect changes in mitochondrial polarization. PA myocytes on collagen-coated cover slips are loaded with TMRE (100 nM) for 45 minutes at 37° C. The cells are then placed in a flow-through chamber on an inverted microscope and perfused with M199 media without serum containing TMRE (10 nM). Fluorescence images are obtained under baseline normoxic conditions (16% O₂/5% CO₂) until a stable baseline is established. The PO₂ of the media is then lowered for 30 mm (1% O₂/5% CO₂), after which it is restored to normoxia for 30 minutes. Finally, to confirm sensitivity to changes in ΔΨm, the protonophore carbonyl cyanide p-trifluoromethoxyphenylhydrazone (FCCP, 10 μM) is added to dissipate the proton gradient across the inner membrane. Hypoxia did not induce a significant change in ΔΨm, nor did ΔΨm change after return to normoxia. However, a rapid, significant decrease in fluorescence is observed upon addition of FCCP. These findings indicate that mitochondrial permeability transition pore opening did not occur during hypoxia or after return to normoxia.

Protein nitrosylation assays used NITRIGLO, Perkin-Elmer Life Science, for both the isolated myocytes and endothelia.

Cytokines assays are as described by Ahmad M et al. (peds research 1998). Microassay (U-Vision Biotech) for endothelia isolation and Microarray analysis, Deptartment of Biochemistry, Michigan State University for the pulmonary myocyte isolation.

TNF-αassay is as follows. Arterial blood samples of 1.8 mL obtained at each time point were mixed with 0.2 mL cold inhibitor solution containing indomethacin and sodium EDT A, and centrifuged (10,000 rpm) at 4° C. for 10 min. The serum was decanted and stored at −70° C. until a TNF ELISA was performed (TNF ELISA kit was provided by Genentech, Inc., South San Francisco, Calif.). The ELISA was performed as previously described ______. The piglet serum TNF levels were derived from the average of 2 assays per sample. The lower limit of TNF detection by this assay was about 35 μg/mL.

(II) Meconium Induced Pulmonary Hypertension:

The above method using isolated perfused lung is repeated in normoxia, using fine granulated meconium as described by Lu ______ (2000) and using IκB-β instead of IκB-α.

First-passed meconium is taken from urine-free diapers of normal term infants in well-baby nursery. The meconium is placed in a sterile jar and frozen for less than 7 days and then lyophilized. Pooling meconium from three to six infants minimized possible variability. Dry meconium is mixed in sterile water by vortex at a concentration of 30 mg/mL, and then aspirated into a tuberculin syringe through a 25-gauge needle. Five mL/kg body weight is instilled via the tracheostomy in less than 10 seconds. Half the dose is given with the rat lying on one side and half with the rat lying on the other side. Five minutes after giving the meconium, furosemide, 10 mg/kg, is injected via the carotid artery to reduce fluid retention in the lungs.

Blood gas determinations are recorded approximately 15 min after the meconium injury. Repeat doses of meconium are administered (1 mL/kg) if the Pa_{O2} is above 115 mm Hg.

Sixty minutes after the last dose of meconium, pulmonary microvessels myocytes and endothelia are isolated as mentioned above and treatment is given.

(III) LPS Induced Pulmonary Hypertension:

The above method using isolated perfused lung is repeated in normoxia using IKB-β instead of IκB-α, and using LPS to induce pulmonary hypertension as mentioned in meconium section.

(IV) GBS-Induced Pulmonary Hypertension:

As in LPS section.

Group B steptococcus (GBS) preparation is as follows. A clinical isolate of type III Group B (3-hemolytic streptococci (COH-I) is prepared as previously described in Pauly ______ (1992). Bacteria were incubated in Todd-Hewitt broth for 18 hour prior to each experiment. The broth culture was then centrifuged (3,000 rpm) at 4° C. for 10 minutes, washed, and resuspended in non-bacteriostatic normal saline. Final concentration of bacteria was determined by optical density, using a previously determined plot associating optical density with bacteria colony forming units (cfu)/mL.

Experimental Protocol is as follows. Fifteen rats were randomly assigned to 1 of 3 groups. Measurements taken at each time point included arterial and mixed venous blood gases, inert gas determinations in samples of arterial blood, mixed venous blood and expired gas, and TNF and TxB2 assays, mean P sa′ mean PAP, P pcw′ and CO from arterial blood, and PVR (PVR=PAP−P pcw/CO), and systemic vascular resistance (SVR=P sa/CO) were calculated at each time point.

After baseline measurements, all rats received continuous infusion of GBS (1.25×109 cfu/kg/hr) for 5 hours. Repeat measurements were made at 0.5, 4.0, 4.5, and 5.0 hr of GBS infusion. After 4 hours of GBS infusion, treated animals received amr (amr 10 and amr 20) (Inocor lactate, Sterling Drug Co., New York, N.Y.); control animals (control) received the amr carrier solution containing lactic acid and sodium metabisulfite. Amr 10 rats (n=5; 11.2: t 4.1 days; 3.0: t 0.5 kg) received an amr bolus (8 mg/kg) followed by a continuous infusion of amr diluted with normal saline (10 about g/kg/min) for 1 hour. Amr 20 rats (n=5; 9.2 t 2.2 days; 2.2: t 0.4 kg) received the same amr bolus (8 mg/kg) followed by a continuous infusion of amr diluted with normal saline (20 about g/kg/min) for 1 hour. Control rats (n=5; 11.0: t 1.4 days; 2.7: t 0.4 kg) received a bolus of the amr carrier solution containing lactic acid (8 mg/kg) and sodium metabisulfite (0.4 mg/kg) followed by a 1 hour infusion of amr carrier diluted with saline. The dosage and concentration of lactic acid were 0.6 mg/kg and 0.33 mg/mol, respectively, and of sodium metabisulfite, 0.03 fig/kg and 0.017 mg/fiL, respectively. Treated and control animals had similar bolus and infusion volumes (1.6 mud kg and 1.8 mUkg/h, respectively) to control for volume effect on homodynamic.

Upon completion of studies on these 3 groups of animals, a fourth group of rats was studied (n=3; 10.3: t 2.7 days; 2.6: t 0.4 kg) to determine if a higher infusion rate of arnr could sustain a selective pulmonary vasodilator effect. These rats were under the same experimental protocol as the other arnr treated groups with the exception of receiving a I h arnr infusion of 40 about g/kg/min (arnr 40). The dosage for the afir bolus given to all treated groups was based on pilot experiments, which demonstrated that an 8 fig/kg bolus of afir would induce an acute reduction in elevated PVR. The arnr infusion dosages were chosen from human studies which demonstrated arnr to be successful in reversing non-septic forms of pulmonary hypertension at infusion rates of 10 to 40 about g/kg/min. All rats were sacrificed after 5 hours of GBS infusion by an overdose of pentobarbital followed by KCl.

After pulmonary hypertension has been induced, the lungs are treated with the composition of the present invention and the decrease in pulmonary hypertension ascertained.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

I claim:

1- A method for treating pulmonary hypertension in a patient, which comprises:
(a) providing a composition comprising a vasodilator, an antioxidant, and at least one chemical selected from the group consisting of endothelin-1 receptor antagonists and antithromboxanes in a mixture with a pulmonary or lung surfactant; and
(b) administering the composition intratracheally to the patient to treat the pulmonary hypertension.

2- The method of claim 1 wherein the vasodilator is a long acting nitric oxide donor.

3- The method of claim 2 wherein the vasodilator is selected from the group consisting of isosorbide dinitrate, hydralazine, minoxidil, nitric oxide gas, and combinations thereof.

4- The method of claim 1 wherein the antioxidant is coenzyme Q₁₀.

5- The method of claim 1 wherein the antithromboxane is selected from the group consisting of prostacyclin, and sulotroban.

6- The method of claim 1 wherein the patient is an infant.

7- A method for treating pulmonary hypertension in a patient, which comprises:
(a) providing a composition comprising a vasodilator, an antioxidant, an endothelin-1 receptor antagonist, and an antithromboxane in a mixture with a pulmonary or lung surfactant; and
(b) administering the composition intratracheally to the patient to treat the pulmonary hypertension.

8- The method of claim 7 wherein the vasodilator is a long acting nitric oxide donor.

9- The method of claim 8 wherein the vasodilator is selected from the group consisting of isosorbide dinitrate, hydralazine, minoxidil, nitric oxide gas, and combinations thereof.

10- The method of claim 7 wherein the antioxidant is coenzyme Q₁₀.

11- The method of claim 7 wherein the antithromboxane is selected from the group consisting of prostacyclin, and sulotroban.

12- The method of claim 7 wherein the patient is an infant.

13- A composition comprising:
a vasodilator, an antioxidant, and at least one chemical selected from the group consisting of endothelin-1 receptor antagonists and antithromboxanes in a mixture with a pulmonary or lung surfactant.

14- The composition of claim 13 wherein the vasodilator is a long acting nitric oxide donor.

15- The composition of claim 14 wherein the vasodilator is selected from the group consisting of isosorbide dinitrate, hydralazine, minoxidil, nitric oxide gas, and combinations thereof.

16- The composition of claim 13 wherein the antioxidant is coenzyme Q₁₀.

17- The composition of claim 13 wherein the antithromboxane is selected from the group consisting of prostacyclin, and sulotroban.

18- A composition comprising:
a vasodilator, an antioxidant, an endothelin-1 receptor antagonist, and an antithromboxane in a mixture with a pulmonary or lung surfactant.

19- The composition of claim 18 wherein the vasodilator is a long acting nitric oxide donor.

20- The composition of claim 19 wherein the vasodilator is selected from the group consisting of isosorbide dinitrate, hydralazine, minoxidil, nitric oxide gas, and combinations thereof.

21- The composition of claim 18 wherein the antioxidant is coenzyme Q₁₀.

22- The composition of claim 18 wherein the antithromboxane is selected from the group consisting of prostacyclin, and sulotroban.

23- A method for treating pulmonary hypertension in a patient, which comprises:
(a) providing a liposome comprising a pulmonary or lung surfactant and which contains therein a vasodilator and chemicals which modulate development of acute inflammation; and
(b) administering the liposome to the patient intratracheally to treat the pulmonary hypertension.

24- The method of claim 23 wherein the vasodilator is a long-acting nitric oxide donor.

25- The method of claim 23 wherein the chemicals which modulate development of acute inflammation comprise recombinant IκB-α, an inhibitor of activation of NF-κB, anti-TNF-α, and an iNOS inhibitor and peroxynitrite scavenger.

26- The method of claim 25 wherein the anti-TNF-α is a recombinant polypeptide comprising the TNF-α receptor.

27- The method of claim 24 wherein the iNOS inhibitor and peroxynitrite inhibitor is mercaptoethylguanidine.

28- The method of claim 24 wherein the inhibitor of activation of NF-κB is a dithiocarbamate.

29- The method of claim 24 wherein the liposome is mixed with a composition which comprises a vasodilator, an antioxidant, an endothelin-1 receptor antagonist, and an antithromboxane in a mixture with a pulmonary or lung surfactant.

30- The method of claim 24 wherein the patient is an infant.

31- The method of claim 24 wherein the patient has pulmonary hypertension which does not respond to nitric oxide treatment.

32- A composition which comprises:
a liposome comprising a pulmonary or lung surfactant and which contains therein a vasodilator and chemicals which modulate development of acute inflammation.

33- The composition of claim 32 wherein the vasodilator is a long-acting nitric oxide donor.

34- The composition of claim 32 wherein the chemicals which modulate development of acute inflammation comprise recombinant IκB-α, an inhibitor of activation of NF-κB, anti-TNF-α, and an iNOS inhibitor and peroxynitrite scavenger.

35- The composition of claim 32 wherein the anti-TNF-α is a recombinant polypeptide comprising the TNF-α receptor.

36- The composition of claim 32 wherein the iNOS inhibitor and peroxynitrite inhibitor is mercaptoethylguanidine.

37- The composition of claim 32 wherein the inhibitor of activation of NF-κB is a dithiocarbamate.

38- The composition of claim 32 wherein the composition further comprises an antioxidant, an endothelin-1 receptor antagonist, and an antithromboxane in a mixture with a pulmonary or lung surfactant.
